(19) United States
(12) Patent Application Publication
(10) Pub. No.: US 2007/0185353 A1
(43) Pub. Date: Aug. 9, 2007

(54) Method for producing alkylene glycol diethers

(75) Inventors: Alexander Snell, Altoetting (DE); Achim Stankowiak, Altoetting (DE); Gabriele Oberendfellner, Burgkirchen (DE); Hieng Kim, Nidderau (DE); Klaus Forstinger, Babenhausen (DE); Christian Wille, Weinheim (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT PRODUKTE (DEUTSCHLAND) GMBH, FRANKFURT AM MAIN (DE)

(21) Appl. No.: 10/590,187

(22) PCT Filed: Feb. 8, 2005
(86) PCT No.: PCT/EP05/01236

§ 371 Date: Aug. 17, 2006

(30) Foreign Application Priority Data
Feb. 17, 2004 (DE) 10 2004 007 561.1

(51) Int. Cl. C07C 41/01

(52) U.S. Cl. 568/579

(57) ABSTRACT

The invention relates to a method for producing alkylene glycol diethers by reacting a linear or cyclic ether with an alkylene oxide in the presence of a Lewis acid. The invention is characterized in that the reaction is continuously carried out in a micro reactor.

The present invention relates to a process for preparing catenary alkylene glycol diethers in a microreactor.

Alkylene glycol diethers are widely used as polar inert solvents. Processes described or industrially practiced for their manufacture include not only indirect processes such as, for example, Williamson's ether synthesis (K. Weissermel, H. J. Arpe “Industrielle Organische Chemie”, 1998, page 179) or the hydrogenation of diglycol ether formal (DE-A-24 34 057), but also direct processes such as, for example, the insertion of alkylene oxide into a catenary ether in the presence of Lewis acids such as BF₃ (U.S. Pat. No. 4,146,736 and DE-A-26 40 505 in conjunction with DE-A-31 28 962) or SnCl₄ (DE-A-30 25 434).

To achieve consistent product quality, it is necessary to police process parameters such as temperature, time and degree of commixing.

DD 246 257 A1 discloses that miniaturized engineering apparatus can be used for chemical reactions. It is known to conduct certain chemical conversions in microreactors. The term microreactor herein shall also comprehend minireactors, which differ from microreactors in terms of the dimensions and constructions of the microstructured reaction channels. The term microreactor shall also comprehend a combination of a static micromixer with an attached temperature-controllable delay sector (a continuous tubular reactor), for example a capillary.

Microreactors are constructed of stacks of structured laminae and are described in DE 39 26 466 C2 for example.

The literature knows of various microreactor-based manufacturing processes (cf. Ullmann's Encyclopedia 2003 of Industrial Chemistry, 6th edition, CD-ROM 2003). Microreactor processes have been described for preparing ethylene oxide (Ing. Eng. Chem. Res. 2002, 41, 710) and the conversion of ethylene oxide to monoethylene glycol (U.S. Pat. No. 4,760,200; U.S. Pat. No. 4,579,982). A process for preparing catenary alkylene glycol diethers in a microreactor is not known.

The present invention therefore has for its object to provide a process for preparing catenary alkylene glycol diethers that allows efficient control of process parameters for the purpose of achieving consistent product quality. The process should further allow improved plant safety and for simple, quick scale-up from the laboratory scale to an industrial scale.

The present invention relates to the use of a microreactor for preparing alkylene glycol diethers by a direct Lewis acid catalyzed process under pressure. An advantage over prior art processes is the simple and inexpensive possibility of plant expansion. In addition, a microreactor offers a high degree of safety (low molecular weight alkylene glycol diethers such as monoethylene glycol dimethyl ether for example are toxic and carcinogenic), since the reaction volume in a microreactor is particularly small compared with conventional batch processes.

We have found that this object is achieved by a process for preparing an alkylene glycol diether by reacting a linear or cyclic ether with an alkylene oxide in the presence of a Lewis acid, wherein the reaction is carried out continuously in a microreactor.

DE-A-3128962 discloses that an only sparingly soluble oxonium salt is formed when BF₃ is used as Lewis acid catalyst. It is pointed out in U.S. Pat. No. 5,811,062 that microreactors are preferably used for reactions that do not require materials or solids that would clog the microchannels and that do not produce materials or solids that would clog the microchannels.

We have now found that, surprisingly, the production of alkylene glycol diethers under Lewis acid catalysis is possible under the conditions described in this invention even though it has hitherto been assumed that the generation of solids (oxonium salt or Lewis acid) in a microreactor would cause the latter to become clogged.

In the process of the present invention, the linear or cyclic ethers, the alkylene oxide and also the requisite Lewis acid are metered into the reactor in liquid form (under pressure if necessary). The rates of addition are controlled for example via mass flow meters or a gravimetric metering control system. The reaction is carried out at a pressure in the range from 0 to 30 bar (above atmospheric pressure), preferably at a pressure in the range from 8 to 20 bar, and at a temperature in the range from 0° C. to 200° C. and preferably in the range from 20° C. to 1 50° C. After the reaction of the reactants, the reaction mixture comprising the product which has formed is brought to atmospheric pressure via a depressurizing vessel and subsequently worked up.

Ethers useful as starting materials for the process of the present invention include various ethers having lower alkyl groups, especially those of the general formula I:
R¹—O—R²
where R¹ is C₁ to C₁₂ alkyl, R² is C₁ to C₁₂ alkyl, phenyl or benzyl or wherein R¹ and R² combine to form a ring of 5, 6 or 7 atoms that encloses the oxygen atom.

Preferably, R¹ and R² are independently C₁ to C₄ alkyl, in particular methyl or ethyl.

A ring formed by R¹ and R² conforms to the formula 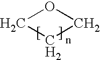
where n is 2, 3 or 4. Tetrahydrofuran is a preferred cyclic compound.

Various alkylene oxides can be used in the present invention. Preference is given to the compounds of the general formula II 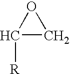
where R is hydrogen, halogen, alkyl having 1 to 10 carbon atoms, phenyl or benzyl.

Examples of suitable alkylene oxides are ethylene oxide, propylene oxide, butylene oxide, epichlorohydrin, styrene oxide and a mixture thereof. Ethylene oxide and propylene oxide are particularly preferred.

The compounds obtained by the process of the present invention conform to the formula
R¹—O—[—(CH₂)ₓ—O]_{y}—R²
where independently
R¹ is C₁ to C₁₂ alkyl
R² is C₁ to C₁₂ alkyl, phenyl or benzyl,
x is an integer from 1 to 6
y is an integer from 1 to 20.

Preferably, R¹ and R² are each methyl or ethyl, in particular methyl.

Useful Lewis acids for the process of the present invention differ very widely in terms of composition and structure.

Preference is given to Lewis acids (individually or combined) in the form of metal or nonmetal halides, for example BF₃, AlCl₃, FeCl₃, SnCl₄, PF₅, SbF₅; in the form of hydrogen acids, for example HBF₄, HBO₂; in the form of heteropolyacids such as for example tungsten heteropolyacid; in the form of coordination complexes of metal and nonmetal halides with organic compounds, for example haloalkyls, ethers, acid chlorides, acid esters or acid anhydrides. Also suitable are trialkyloxonium salt complexes having identical or different alkyl groups, analogous acylium salt complexes and also unsaturated tertiary oxonium salts, the tertiary carboxonium salts.

Solvents can be employed in the process of the present invention if they offer advantages with regard to the preparation of catalysts, for example for increasing the solubility, and/or for raising/lowering the viscosity and/or for removing heat of reaction. Examples thereof are inert solvents such as dichloromethane, nitromethane, benzene, toluene, acetone, ethyl acetate, or dioxane or active solvents such as for example methanol, ethanol, propanol, butanol, methylglycol, methyldiglycol, methyltriglycol, or the target substances themselves such as mono-, di-, tri-, tetra- or polyalkylene glycol dimethyl ether.

The process of the present invention provides alkylene glycol diethers in good yield in a continuous operation in a microreactor, if appropriate in combination with further batch operating steps (for example preparation of reactant or catalyst mixtures, workup of reaction mixture).

Prior art microreactors may be used, for example commercially available microreactors, for example the Cytos™ based Selecto™ from Cellular Process Chemistry GmbH, Frankfurt/Main.

It is also possible to use microreactors having two or more reaction zones for the individual reaction steps. A microreactor is constructed from a plurality of laminae which are stacked and bonded together and whose surfaces bear micromechanically created structures which cooperate to form reaction spaces in which chemical reactions take place. The system contains at least one continuous channel connected to the inlet and the outlet.

The flow rates for the streams of material are limited by the apparatus, for example by the pressures resulting from the geometry of the microreactor. The flow rates are preferably between 0.05 and 5 l/min, more preferably between 0.05 and 500 ml/min and even more preferably between 0.05 and 250 ml/min.

The reaction channel of a preferred microreactor is a capillary having any desired, for example round, cross section and generally having a diameter in the range from 200 to 2000 μm and preferably in the range from 400 to 1000 μm. Numerous parallelized reaction channels may be provided, if desired, to increase throughput.

The heat exchanger is preferably likewise a capillary having any desired, for example a round, cross section, and generally having a diameter in the range from 200 to 800 μm.

The production of mixtures of feedstocks to form reactant streams can take place beforehand in micromixers or upstream mixing zones. It is also possible for feedstocks to be metered in downstream mixing zones or in downstream micromixers or -reactors.

The preferred microreactor is fabricated from stainless steel; other materials can also be used, examples being glass, ceramic, silicon, plastics or other metals.

A suitable microreactor is depicted in the description part and FIG. 1 of DE-A-100 40 100.

### EXAMPLES

The following abbreviations are used:

DMG=dimethylethylene glycol

DMDG=dimethyldiethylene glycol

DMTG=dimethyltriethylene glycol

DMTeG=dimethyltetraethylene glycol

DMPeG=dimethylpentaethylene glycol

### Examples 1 to 6

At a temperature of 60° C. and variable pressure (see Table 1) 30 g/h (0.68 mol) of ethylene oxide and 315 g/h (6.85 mol) of dimethyl ether are introduced into the microreactor in liquid form. In addition, 0.17 mol of boron fluoride dimethyl etherate dissolved in 500 ml of polyethylene glycol dimethyl ether (average molecular weight 500 g/mol) is pumped into the reactor at a feed rate of 45 ml/h. Following a residence time of 2 min, the reaction mixture is depressurized into a steel collecting vessel and subsequently analyzed by gas chromatography. Thereafter, the mixture is neutralized with NaHCO₃ and worked up by distillation.

TABLE 1

| | | GC analysis [area %] | | | | | | |
|---|---|---|---|---|---|---|---|---|
| No. | p/bar | DMG | DMDG | DMTG | DMTeG | DMPeG | Dioxane | Others |
| 1 | 9 | 19.6 | 30.2 | 9.2 | 3.4 | 0 | 18.4 | 19.2 |
| 2 | 10 | 22.4 | 29.6 | 10.2 | 4.9 | 1.2 | 21.2 | 10.5 |
| 3 | 11 | 20.4 | 26.6 | 11.3 | 5.3 | 0.9 | 28.7 | 6.8 |
| 4 | 12 | 31.2 | 34.8 | 12.7 | 5.3 | 1.7 | 10.1 | 4.2 |
| 5 | 13 | 24.8 | 17.5 | 7.5 | 3.1 | 0 | 33.2 | 13.9 |
| 6 | 14 | 22.8 | 16.1 | 13.2 | 7.6 | 2.6 | 26.3 | 11.4 |

### Examples 7 to 11

Performed similarly to Examples 1 to 6 except that the temperature is varied (see Table 2) while the pressure was kept constant at 12 bar.

TABLE 2

| | | GC analysis [area %] | | | | | | |
|---|---|---|---|---|---|---|---|---|
| No. | T/° C. | DMG | DMDG | DMTG | DMTeG | DMPeG | Dioxane | Others |
| 7 | 50 | 18.6 | 17.4 | 8.7 | 2.5 | 0.8 | 34.1 | 17.9 |
| 8 | 60 | 20.4 | 26.6 | 11.3 | 1.7 | 0 | 33.2 | 6.8 |
| 9 | 70 | 25.8 | 33.6 | 13.8 | 1.9 | 1.1 | 12.0 | 11.8 |
| 10 | 75 | 36.4 | 28.2 | 10.1 | 2.3 | 1.5 | 11.9 | 9.6 |
| 11 | 80 | 31.0 | 32.4 | 15.4 | 2.3 | 1.4 | 8.2 | 9.3 |

### Examples 12 to 14

At a temperature of 60° C. and a pressure of 12 bar 30 g/h (0.68 mol) of ethylene oxide and 315 g/h (6.85 mol) of dimethyl ether are introduced into the microreactor in liquid form. In addition, 0.17 mol of boron fluoride dimethyl etherate dissolved in 500 ml of polyethylene glycol dimethyl ether (average molecular weight 500 g/mol) is pumped into the reactor at a variable feed rate (see Table 3). Following a residence time of 2 min, the reaction mixture is depressurized into a steel collecting vessel and subsequently analyzed by gas chromatography. Thereafter, the mixture is neutralized with NaHCO₃ and worked up by distillation.

TABLE 3

| | | | GC analysis [area %] | | | | | | |
|---|---|---|---|---|---|---|---|---|---|
| No. | Feed rate | | DMG | DMDG | DMTG | DMTeG | DMPeG | Dioxane | Others |
| 12 | 45 | g/h | 21.8 | 24.6 | 10.6 | 3.1 | 2.1 | 25.2 | 12.6 |
| 13 | 90 | g/h | 25.4 | 28.0 | 13.7 | 5.4 | 1.9 | 16.4 | 9.2 |
| 14 | 180 | g/h | 27.8 | 15.6 | 14.2 | 4.8 | 2.3 | 29.5 | 5.8 |

1. A process for preparing an alkylene glycol diether by reacting a linear or cyclic ether with an alkylene oxide in the presence of a Lewis acid, wherein the reaction is carried out continuously in a microreactor.

2. The process according to claim 1 wherein the alkylene glycol diether conforms to the formula
R¹—O—R²
where R¹ is C₁ to C₁₂ alkyl, R² is C₁ to C₁₂ alkyl, phenyl or benzyl or wherein R¹ and R² combine to form a ring of 5, 6 or 7 atoms that encloses the oxygen atom.

3. The process according to claim 1, wherein the alkylene oxide conforms to the formula 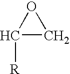
where R is H, halogen, C₁ to C₁₀ alkyl, phenyl or benzyl.

4. The process of claim 1, wherein the Lewis acid is selected from the group consisting of metal and nonmetal halides, hydrogen acids, heteropolyacids, haloalkyls, ethers, acid chlorides, acid esters, acid anhydrides, trialkyloxonium salt complexes having identical or different alkyl groups, acylium salt complexes, unsaturated tertiary oxonium salts, and mixtures thereof.

5. The process of claim 1, wherein the reacting step is carried out in a solvent selected from the group consisting of dichloromethane, nitromethane, benzene, toluene, acetone, ethyl acetate, dioxane, methanol, ethanol, propanol, butanol, methylglycol, methyldiglycol, methyltriglycol, mono-glycol dimethyl ether, polyalkylene glycol dimethyl ether, and mixtures thereof.

6. The process of claim 1, wherein the microreactor has a reaction channel which is a capillary having a round cross section, said capillary having a diameter in the range from 400 to 1000 μm.
